(19) United States
(12) Patent Application Publication
(10) Pub. No.: US 2013/0030288 A1
(43) Pub. Date: Jan. 31, 2013

(54) IMAGE DIAGNOSIS APPARATUS INCLUDING X-RAY IMAGE TOMOSYNTHESIS DEVICE AND PHOTOACOUSTIC IMAGE DEVICE AND IMAGE DIAGNOSIS METHOD USING THE SAME

(71) Applicants: Jeong Won LEE, Daejeon (KR); Chang-Geun AHN, Daejeon (KR); Sooyeul LEE, Daejeon (KR); Donghoon YU, Daejeon (KR); Seunghwan KIM, Daejeon (KR)

(72) Inventors: Jeong Won LEE, Daejeon (KR); Chang-Geun AHN, Daejeon (KR); Sooyeul LEE, Daejeon (KR); Donghoon YU, Daejeon (KR); Seunghwan KIM, Daejeon (KR)

(73) Assignee: Electronics and Telecommunications Research Institute, Daejeon (KR)

(21) Appl. No.: 13/557,126

(22) Filed: Jul. 24, 2012

(30) Foreign Application Priority Data
Jul. 28, 2011 (KR) 10-2011-0075414
May 18, 2012 (KR) 10-2012-0053275

(51) Int. Cl. A61B 6/02

(52) U.S. Cl. 600/427

(57) ABSTRACT

Provided are an image diagnosis apparatus and an image diagnosis method using the same. The image diagnosis apparatus includes an X-ray image tomosynthesis device fixing an object using an object fixing unit, the X-ray image tomosynthesis device generating an image tomosynthesis signal by irradiating a plurality of X-rays into the object, a photoacoustic image device fixing the object using the object fixing unit, the photoacoustic image device generating an ultrasonic signal by scanning the object using a photoacoustic light source, an image processing device for processing image signals transmitted from the X-ray image tomosynthesis device and the photoacoustic image device to generate a three-dimensional (3D) image, and a display device for displaying the 3D image generated from the image processing device. The image diagnosis apparatus may realize internal tissues of the object into a clear 3D image.

### CROSS-REFERENCE TO RELATED APPLICATIONS

This U.S. non-provisional patent application claims priority under 35 U.S.C. §119 of Korean Patent Application Nos. 10-2011-0075414, filed on Jul. 28, 2011, and 10-2012-0053275, filed on May 18, 2012, the entire contents of which are hereby incorporated by reference.

### BACKGROUND OF THE INVENTION

The present invention relates to an image diagnosis apparatus, and more particularly, to an image diagnosis apparatus including an X-ray image tomosynthesis device and a photoacoustic image device and an image diagnosis method using the same.

Various image diagnosis apparatus are being used for medical image diagnosis. For example, computed tomography (CT) systems or nondestructive inspectors are used for detecting cancer tissues. The CT systems may be effectively used for diagnosing a breast cancer because cancer tissues are hard tissues. For example, a density change image of a material composing an object (the breast) may be acquired by pressing the breast in up and down or the inside and outside directions to detect cancer tissues.

Also, the nondestructive inspection using an X-ray may be used for detecting cancers. One of the main purposes of the use of the X-ray is to detect cancers. For this, a system having high sensitivity may be required. However, in actual clinical tests, high specificity as well as the high sensitivity is required also in the system.

### SUMMARY OF THE INVENTION

The present invention provides an image diagnosis apparatus and method which may realize internal tissues of an object into a clear three-dimensional (3D) image.

Embodiments of the present invention provide image diagnosis apparatuses including: an X-ray image tomosynthesis device fixing an object using an object fixing unit, the X-ray image tomosynthesis device generating an image tomosynthesis signal by irradiating a plurality of X-rays into the object; a photoacoustic image device fixing the object using the object fixing unit, the photoacoustic image device generating an ultrasonic signal by scanning the object using a photoacoustic light source; an image processing device for processing image signals transmitted from the X-ray image tomosynthesis device and the photoacoustic image device to generate a three-dimensional (3D) image; and a display device for displaying the vivid 3D image on the image processing device.

In some embodiments, the X-ray image tomosynthesis device may include: an X-ray source for irradiating the plurality of X-rays; the object fixing unit for fixing the object; and an X-ray detection unit for detecting the image tomosynthesis signal.

In other embodiments, wherein the object fixing unit may include: a hard compressor for mounting the object thereon; and a soft compressor for covering the object to apply a pressure in a predetermined direction.

In still other embodiments, the object fixing unit may further include a controller for adjusting positions of the hard compressor and the soft compressor.

In even other embodiments, the controller may adjust a position of the X-ray source.

In yet other embodiments, the X-ray detection unit may be disposed on a side opposite to that of the X-ray source with the object fixing unit therebetween.

In further embodiments, the photoacoustic image device may include: the object fixing unit for fixing the object; and an ultrasonic detection unit for detecting the ultrasonic signal.

In still further embodiments, the object fixing unit and the ultrasonic detection unit may contact each other.

In even further embodiments, the photoacoustic light source and the ultrasonic detection unit may be disposed on sides opposite to each other with the object fixing unit therebetween.

In yet further embodiments, the photoacoustic light source and the ultrasonic detection unit may be disposed on the same side as each other with respect to the object fixing unit.

In much further embodiments, the image processing device may include: an X-ray 3D image reconstruction unit for reconstructing the image signal transmitted from the X-ray detection unit to generate the 3D image; a photoacoustic tomography (PAT) 3D image reconstruction unit for reconstructing the image signal transmitted from the ultrasonic detection unit to generate the 3D image; and an image registration unit for registering the 3D images transmitted from the X-ray 3D image reconstruction unit and the PAT 3D image reconstruction unit to generate a 3D image with respect to the object.

In other embodiments of the present invention, image diagnosis methods using an image diagnosis apparatus include: irradiating a plurality of X-rays on an object in a state where the object is fixed using an object fixing unit to generate an image tomosynthesis signal; scanning the object using a photoacoustic light source in the state where the object is fixed to generate an ultrasonic signal; and processing the image tomosynthesis signal and the ultrasonic signal to generate a three-dimensional (3D) image.

In some embodiments, image diagnosis methods may further include searching whether a region of interest (ROI) exists using the generated image tomosynthesis signal.

In other embodiments, when the ROI exists, the photoacoustic light source may scan the ROI.

In still other embodiments, when the ROI does not exist, the photoacoustic light source may scan an entire region of the object.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is a block diagram of an image diagnosis apparatus according to an embodiment of the present invention;

FIG. 2 is a view illustrating an example of an object fixing unit of FIG. 1;

FIG. 3 is a conceptual view for explaining a photoacoustic tomography (PAT) image device of FIG. 1;

FIG. 4 is a block diagram illustrating an image processing device of FIG. 1; and

FIG. 5 is a flowchart for explaining an operation process of the image diagnosis apparatus of FIG. 1.

### DETAILED DESCRIPTION OF PREFERRED EMBODIMENTS

Preferred embodiments of the inventive concept will be described below in more detail with reference to the accompanying drawings, such that those skilled in the art can realizes the technical ideas of the inventive concept without difficulties.

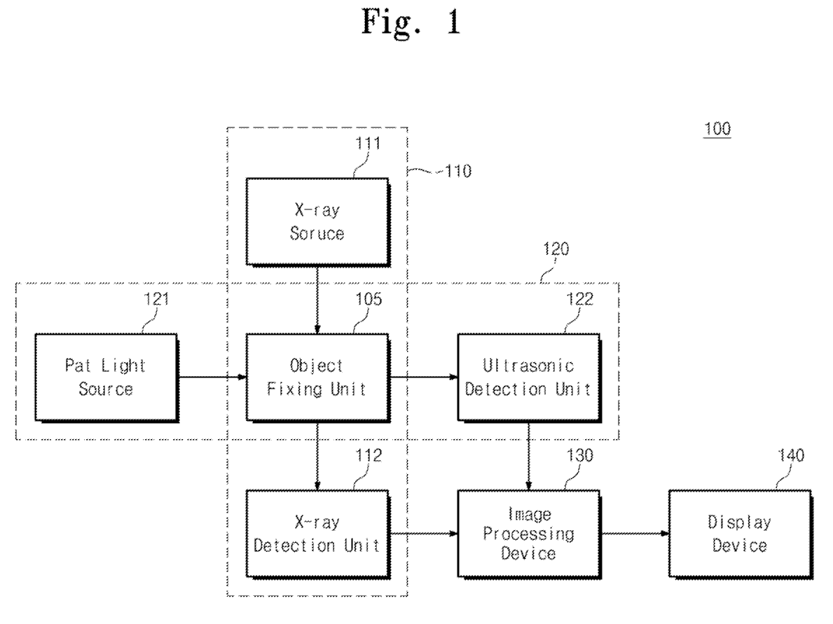

FIG. 1 is a block diagram of an image diagnosis apparatus according to an embodiment of the present invention. The image diagnosis apparatus is an apparatus for imaging the inside of an object to be diagnosed (hereinafter, referred to as an “object”) to output the clearest image by combining an X-ray digital tomosynthesis method and a photoacoustic method. The image diagnosis apparatus may be mainly utilized as a use of accurate biopsy for a portion (e.g., a breast, and the like) of the human body.

The X-ray image tomosynthesis method is a diagnosis method for imaging the inside of the human body using an X-ray source and a detection unit. The X-ray source and the detection unit are rotated around the object to acquire several sheets of projection images, thereby reconstructing the acquired projection images into a three-dimensional (3D) image by using a mathematical technique.

The photoacoustic method is a diagnosis method for imaging the inside of the human body using a photoacoustic tomography (PAT) light source and an ultrasonic detection unit. When light is irradiated into the human body from a light source, tissues within the human body absorb energy to emit supersonic waves. Here, since the tissues have light absorption properties different from each other, the inside of the human body may be three-dimensionally imaged by calculating the intensities and positions of ultrasonic signals.

Referring to FIG. 1, an image diagnosis apparatus 100 includes an X-ray image tomosynthesis device 110, a photoacoustic image device 120, an image processing device 130, and a display device 140. The X-ray image tomosynthesis device 110 shares an object fixing unit 105 with the photoacoustic image device 120.

The X-ray image tomosynthesis device 110 includes the object fixing unit 105, an X-ray source 111, and an X-ray detection unit 112. The X-ray image tomosynthesis device 110 of FIG. 1 may use at least one or more X-ray sources 111, at least one or more X-ray detection units 112, or at least one or more X-ray sources 111 and X-ray detection units 112.

Also, the X-ray image tomosynthesis device 110 of FIG. 1 may be realized as one of a radiography system, a tomosynthesis system, a computed tomography (CT) system, and a non-destructed inspector. However, the above-described devices are exemplified merely for the X-ray image tomosynthesis device 110. Thus, it is obvious to a person skilled in the art to embody the image diagnosis apparatus using an X-ray for various modifications and application examples.

Referring again to FIG. 1, the object fixing unit 105 fixes an object (e.g., a portion of the human body) so that the object is not moved while photographing. The object fixing unit 105 may be designed so that two compression plates press the object in up and down directions, respectively.

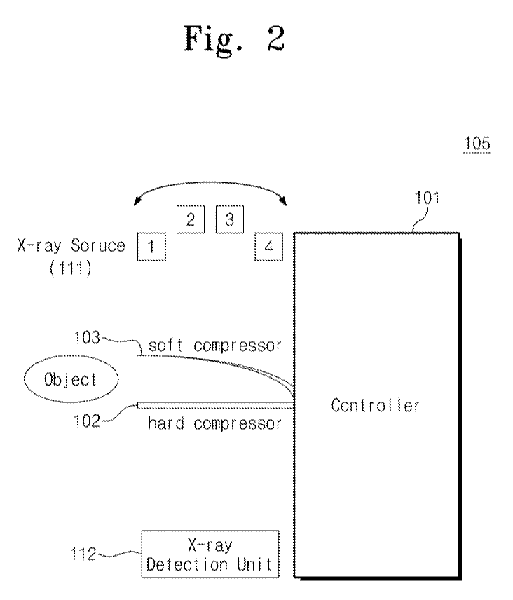

The compression plates (see FIG. 2) may fix the object and also press the object at a predetermined pressure in a predetermined direction when it is necessary to compress the object. Also, the compression plates may be designed so that the compression plates release the pressure applied to the object thereby. The object fixing unit 105 will be described in more detail with reference to FIG. 2.

It is unnecessary that the X-ray source 111 contacts the object fixing unit 105, and the X-ray source 111 irradiates an X-ray into the object. The X-ray irradiated form the X-ray source 111 may include photons having a plurality of energy levels.

The X-ray passing through the object is detected by the X-ray detection unit 112. The X-ray detection unit 112 is disposed on a side opposite to that of the X-ray source 111 with the object fixing unit 105 therebetween. It is unnecessary that the X-ray detection unit 112 contacts the object fixing unit 105. Alternatively, the X-ray detection unit 112 may contact the object fixing unit 105.

The X-ray detection unit 112 may acquire a plurality of images which are produced by the X-ray irradiated from the X-ray source 111 to pass through the object. Particularly, the X-ray detection unit 112 may detect X-ray photons, which are incident from the X-ray source 111 by passing through the object, to acquire a plurality of images. By using an image generated from the plurality of images acquired by compressing the object, hard tissues and soft tissues of the human body may be confirmed, and thus, cancer tissues generated in the human body may be confirmed by confirming the hard tissues.

The tissues composing the object may be largely classified into hard tissues and soft tissues. The hard tissues are hard tissues such as bones. If the hard tissues exist, an image may be degraded in quality due to an overlapping phenomenon of other tissues disposed at a rear side of each of the hard tissues. Also, in a case where the hard tissues are bone tissues, it may be difficult to completely solve the overlapping phenomenon because of the nonuniform composition ratio of the bone tissues.

The image diagnosis apparatus 100 according to an embodiment of the present invention may combine the X-ray image tomosynthesis device 110 with the photoacoustic image device 120 to acquire a more accurate image with respect to the internal tissues of the object. Referring again to FIG. 1, the photoacoustic image device 120 is coupled to the X-ray image tomosynthesis device 110 through the object fixing unit 105. Also, the photoacoustic image device 120 includes a PAT light source 121 and an ultrasonic detection unit 122.

The PAT light source 121 is a unit for transmitting energy into the object to generate ultrasonic waves. The PAT light source 121 irradiates laser light having a short pulse into the object (e.g. tissues of humans or animals). The laser energy is absorbed into the tissues within the object to cause significant temperature increase and thermal expansion. Due to the thermal expansion, ultrasonic waves occur in the object. Since the tissues have light absorption properties different from each other, the inside of the object may be three-dimensionally imaged by calculating the intensities and positions of ultrasonic signals. Here, it is unnecessary that the PAT light source 121 contacts the object fixing unit 105.

The ultrasonic detection unit 122 is a unit for detecting the ultrasonic waves generated in the object by the PAT light source 121. The ultrasonic detection unit 122 may contact the object fixing unit 105. The ultrasonic detection unit 122 may be disposed on a side opposite to that of the PAT light source 121 or on the same side as the PAT light source 121 with respect to the object fixing unit 105.

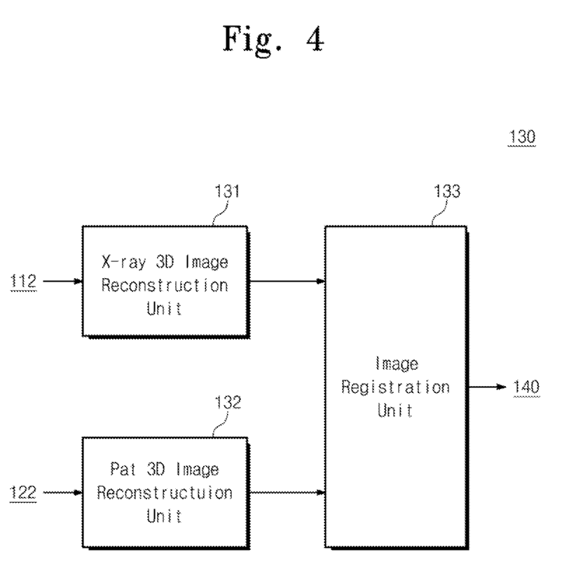

The image processing device 130 generates a 3D image from the plurality of images of the object acquired in the ultrasonic detection unit 122 to perform image processing and synthesis with respect to the generated 3D image. The image synthesized in the image processing device 130 is provided to the display device 140. A constitution and operation method of the image processing device 130 will be described in more detail with reference to FIG. 4.

The image processing device 130 may perform a pre-processing process through an image signal transmitted from the X-ray detection unit 112. For example, the image processing device 30 may previously define a region of interest (ROI) to be inspected in the object. Then, the image processing device 30 searches the ROI in the image to separately store images around the ROI, thereby referring to the stored image when the image is displayed. For another example of the pre-processing process, when the object is a human body, motion artifacts which may occur due to movement of the object during the measurement may be removed.

The image diagnosis apparatus 100 of FIG. 1 may realize the internal tissues of the object into a clear 3D image by successively using the X-ray image tomosynthesis method and the photoacoustic method in a state where the object is fixed using the object fixing unit 105. According to the image diagnosis apparatus 100 of the present invention, it may be possible to accurately take a biopsy of a portion (e.g., a breast, and the like) of the human body.

FIG. 2 is a view illustrating an example of an object fixing unit of FIG. 1.

Referring to FIG. 2, the object fixing unit 105 includes a controller 101, a hard compressor 102, and a soft compressor 103.

The two compression plates may be connected to the controller 101. One of the two compression plates may be the hard compressor 102. Also, the object may be mounted on the hard compressor 102. The X-ray detection unit 112 may be disposed under the hard compressor 102. The other one of the two compression plates may be the soft compressor 103. The soft compressor 103 may cover the object to diagnose the object using the X-ray source 111 or the PAT light source 121. The soft compressor 103 may be manufactured in a mesh shape so that light is easily irradiated into the object or the object is easily scanned.

Referring again to FIG. 2, the controller 101 may control the X-ray source 111 to irradiate an X-ray into the object for a predetermined time at a preset dose or voltage. Also, the controller 101 may control the X-ray source 111 to irradiate the X-ray into the object in preset directions (positions 1 to 4 directions of FIG. 2). That is, the X-ray source 111 may be changed in position to irradiate the X-ray into the object at various angles. Alternatively, a plurality of X-ray sources may be controlled in timing to control an angle of the X-ray irradiated into the object. In FIG. 2, the X-ray source 111 may irradiate the X-ray at the position 1, and then irradiate the X-ray in an order of the position 2, the position 3, and the position 4.

The controller 101 may control the compression plates 102 and 103 to correspond to the control of the X-ray source 111. That is, the controller 101 may control an irradiation angle and time of the X-ray and a degree of the compression of the object.

Although not shown, the controller 101 may control the PAT light source (see reference numeral 121 of FIG. 1) and the ultrasonic detection unit (see reference numeral 122 of FIG. 1). That is, the controller 101 may control the PAT light source 121 to irradiate the X-ray into the object or control an image signal generation of the ultrasonic detection unit 122.

In the case where the photoacoustic method is used, the controller 101 may also control the hard compressor 102 and the soft compressor 103 to fix the object. Here, when the X-ray image tomosynthesis method and the photoacoustic method are used to acquire the same image coordinate with respect to the object, the controller 101 may control the two compression plates 102 and 103 to prevent the object from being moved.

The compression plates 102 and 103 may compress the object to correspond to the irradiated X-ray. In the compression plates 102 and 103, the object may be mounted on the hard compressor 102, and then compressed in a plurality of directions by using the soft compressor 103. The soft compressor 103 may be formed of a deformable material to compress the object.

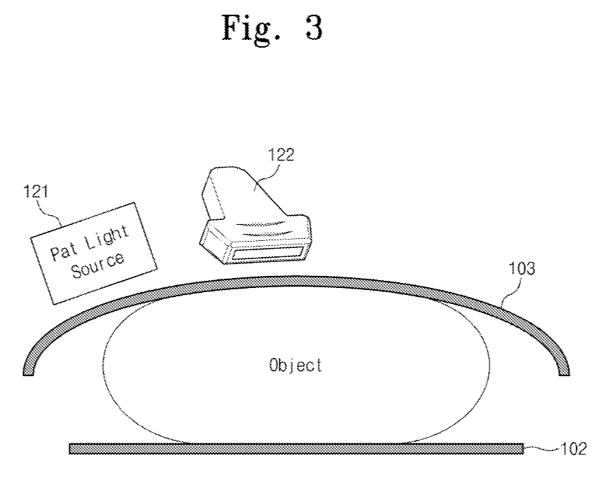

FIG. 3 is a conceptual view for explaining the photoacoustic image device of FIG. 1. Referring to FIG. 3, the object is disposed between the hard compressor 102 and the soft compressor 103. The soft compressor 103 compresses the object so that the PAT light source 121 sufficiently scans the object. An ultrasonic signal generated in the object is detected by the ultrasonic detection unit 122. FIG. 3 illustrates an example of a case in which the ultrasonic detection unit 122 is disposed on the same side as the PAT light source 121 with respect to the object. However, the present invention is not limited thereto. For example, the ultrasonic detection unit 122 may be disposed on a side opposite to that of the PAT light source 121.

FIG. 4 is a block diagram illustrating the image processing device of FIG. 1.

Referring to FIG. 4, the image processing device 130 includes an X-ray 3D image reconstruction unit 131, a PAT 3D image reconstruction unit 132, and an image registration unit 133.

The X-ray 3D image reconstruction unit 131 reconstructs an image signal transmitted from the X-ray detection unit 112 to generate a 3D image. The X-ray 3D image reconstruction unit 131 may generate a 3D image with respect to the object by using a plurality of collected various images. Also, the X-ray 3D image reconstruction unit 131 may acquire a plurality of high-quality images through the compressed degree of the object and a change in position of the X-ray source 111. Also, since the high-quality images are acquired to generate the image with respect to the object, lesions may be accurately and quickly diagnosed.

Similarly, the PAT 3D image reconstruction unit 132 reconstructs an image signal transmitted from the ultrasonic detection unit 122 to generate a 3D image. The PAT 3D image reconstruction unit 132 may generate a 3D image with respect to the object by using a plurality of collected various images. The reconstructed thee-dimensional image is provided to the image registration unit 133.

The image registration unit 133 may register the 3D images transmitted from the X-ray 3D image reconstruction unit 131 and the PAT 3D image reconstruction unit 132 to generate a final image for displaying biopsy results of the object. The final image generated in the image registration unit 133 is provided to the display device 140.

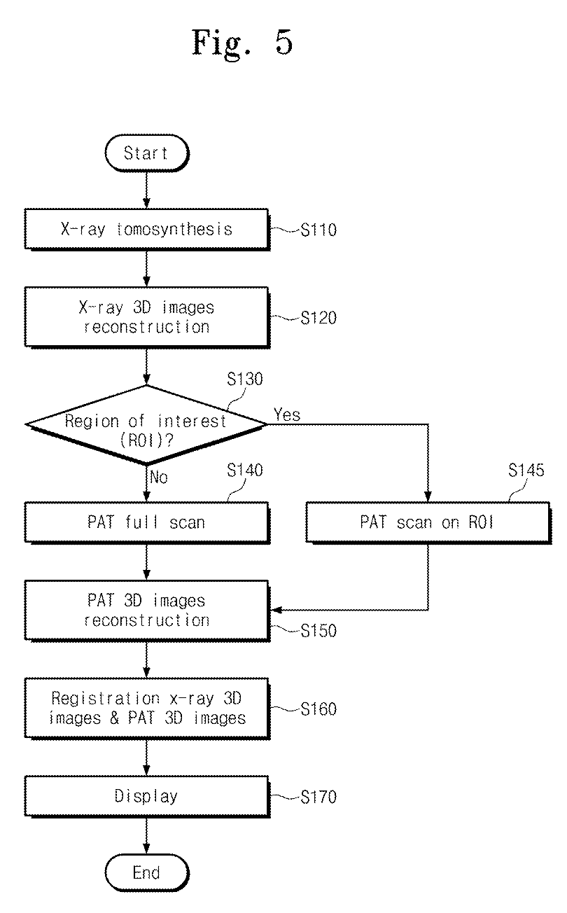

FIG. 5 is a flowchart for explaining an operation process of the image diagnosis apparatus of FIG. 1. Hereinafter, an operation method of the image diagnosis apparatus according to an embodiment of the present invention will be described with reference to FIGS. 1 to 5.

In operation S110, an image signal is generated according to an X-ray image tomosynthesis method. The X-ray image tomosynthesis device 110 compresses the object using the compression plates (see reference numerals 102 and 103 of FIG. 2) to irradiate an X-ray into the compressed object using the X-ray source (see reference numeral 111 of FIG. 1). Here, the X-ray may be irradiated at various angles with respect to the object. The X-ray detection unit (see reference numeral 112 of FIG. 1) may detect X-ray photons which are incident from the X-ray source 111 by passing through the object, to acquire a plurality of images.

In operation S120, the image signal transmitted from the X-ray detection unit 112 is reconstructed to generate a 3D image. The X-ray 3D image reconstruction unit (see reference numeral 131 of FIG. 4) may acquire a plurality of high-quality images through the compressed degree of the object and a change in position of the X-ray source 111.

In operation S130, whether the ROI exists in the X-ray 3D image is searched.

When the ROI exists, a PAT scanning operation is performed on only the ROI in operation S145. Thus, since the ROI is searched, a PAT scanning time may be reduced. If a specific ROI does not exist, the PAT scanning operation is performed on an entire region of the object in operation S140.

In operation S140, the PAT scanning operation is performed on the entire region of the object. The PAT light source (see reference numeral 121 of FIG. 1) transmits energy into the entire region of the object to generate ultrasonic waves. The ultrasonic detection unit 122 detects the ultrasonic waves generated in the object.

In operation S150, the image signal transmitted from the ultrasonic detection unit 122 is reconstructed to generate a 3D image. The PAT 3D image reconstruction unit 132 may generate a 3D image with respect to the object by using a plurality of collected various images. The reconstructed thee-dimensional image is provided to the image registration unit 133.

In operation S160, the image registration unit 133 registers the 3D images transmitted from the X-ray 3D image reconstruction unit 131 and the PAT 3D image reconstruction unit 132 to generate a final image for displaying biopsy results of the object. In operation S170, the final image generated in image registration unit 133 is provided to the display device 140.

Referring to FIG. 5, in the image diagnosis apparatus (see reference numeral 100 of FIG. 1) according to an embodiment of the present invention, the X-ray source 111 and the X-ray detection unit 112 are operated first to acquire an X-ray image, and then the PAT light source 121 and the ultrasonic detection unit 122 are operated in a state where the object is fixed to acquire a photoacoustic image. This is done for a reason in which the ROI is searched through the X-ray image tomosynthesis method to reduce a PAT scanning time and intensively inspect the ROI.

According to the present invention, the X-ray image tomosynthesis method and the photoacoustic method may be combined with each other to display an anatomical image and a photoacoustic functional image, which are acquired through the X-ray, on one coordinate. Since the anatomical image and the functional image are used for purposes different from each other, all of the two images may be important in medical diagnosis.

However, in a typical image acquirement method, there have been studies about methods for registering the two images because it is difficult to search a position of a lesion in the anatomical image corresponding to a position of the lesion in the functional image. According to the present invention, the position of the object may be fixed in the image acquirement process, and also the two types of image devices may be operated to display an image in which the anatomical image and the functional image overlap each other.

The image diagnosis apparatus according to the present invention may realize the internal tissues of the object into the clear 3D image by successively using the X-ray image tomosynthesis method and the photoacoustic method in a state where the object is fixed using the object fixing unit. According to the image diagnosis apparatus of the present invention, it may be possible to accurately take the biopsy of a portion (e.g., the breast, and the like) of the human body.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. An image diagnosis apparatus comprising:
an X-ray image tomosynthesis device fixing an object using an object fixing unit, the X-ray image tomosynthesis device generating an image tomosynthesis signal by irradiating a plurality of X-rays into the object;
a photoacoustic image device fixing the object using the object fixing unit, the photoacoustic image device generating an ultrasonic signal by scanning the object using a photoacoustic light source;
an image processing device for processing image signals transmitted from the X-ray image tomosynthesis device and the photoacoustic image device to generate a three-dimensional (3D) image; and
a display device for displaying the 3D image generated from the image processing device.

2. The image diagnosis apparatus of claim 1, wherein the X-ray image tomosynthesis device comprises:
an X-ray source for irradiating the plurality of X-rays;
the object fixing unit for fixing the object; and
an X-ray detection unit for detecting the image tomosynthesis signal.

3. The image diagnosis apparatus of claim 2, wherein the object fixing unit comprises:
a hard compressor for mounting the object thereon; and
a soft compressor for covering the object to apply a pressure in a predetermined direction.

4. The image diagnosis apparatus of claim 3, wherein the object fixing unit further comprises a controller for adjusting positions of the hard compressor and the soft compressor.

5. The image diagnosis apparatus of claim 4, wherein the controller adjusts a position of the X-ray source.

6. The image diagnosis apparatus of claim 2, wherein the X-ray detection unit is disposed on a side opposite to that of the X-ray source with the object fixing unit therebetween.

7. The image diagnosis apparatus of claim 2, wherein the photoacoustic image device comprises:
the object fixing unit for fixing the object; and
an ultrasonic detection unit for detecting the ultrasonic signal.

8. The image diagnosis apparatus of claim 7, wherein the object fixing unit and the ultrasonic detection unit contact each other.

9. The image diagnosis apparatus of claim 7, wherein the photoacoustic light source and the ultrasonic detection unit are disposed on sides opposite to each other with the object fixing unit therebetween.

10. The image diagnosis apparatus of claim 7, wherein the photoacoustic light source and the ultrasonic detection unit are disposed on the same side as each other with respect to the object fixing unit.

11. The image diagnosis apparatus of claim 7, wherein the image processing device comprises:
an X-ray 3D image reconstruction unit for reconstructing the image signal transmitted from the X-ray detection unit to generate the 3D image;
a photoacoustic tomography (PAT) 3D image reconstruction unit for reconstructing the image signal transmitted from the ultrasonic detection unit to generate the 3D image; and
an image registration unit for registering the 3D images transmitted from the X-ray 3D image reconstruction unit and the PAT 3D image reconstruction unit to generate a 3D image with respect to the object.

12. An image diagnosis method using an image diagnosis apparatus, the image diagnosis method comprising:
irradiating a plurality of X-rays on an object in a state where the object is fixed using an object fixing unit to generate an image tomosynthesis signal;
scanning the object using a photoacoustic light source in the state where the object is fixed to generate an ultrasonic signal; and
processing the image tomosynthesis signal and the ultrasonic signal to generate a three-dimensional (3D) image.

13. The image diagnosis method of claim 12, further comprising searching whether a region of interest (ROI) exists using the generated image tomosynthesis signal.

14. The image diagnosis method of claim 13, wherein, when the ROI exists, the photoacoustic light source scans the ROI.

15. The image diagnosis method of claim 14, wherein, when the ROI does not exist, the photoacoustic light source scans an entire region of the object.
